(19) United States
(12) Patent Application Publication
(10) Pub. No.: US 2022/0409907 A1
(43) Pub. Date: Dec. 29, 2022

(54) HIBERNATION OF ELECTRONICS IN AN IMPLANTABLE STIMULATOR

(71) Applicant: Medtronic, Inc., Minneapolis, MN (US)

(72) Inventors: Joel A. Anderson, Brooklyn Park, MN (US); Robert A. Corey, Arden Hills, MN (US); Gregory J. Loxtercamp, Edina, MN (US); Forrest C.M. Pape, New Brighton, MN (US)

(21) Appl. No.: 17/809,492

(22) Filed: Jun. 28, 2022

Related U.S. Application Data
(60) Provisional application No. 63/215,813, filed on Jun. 28, 2021.

(51) Int. Cl. A61N 1/378; A61N 1/372

(52) U.S. Cl. CPC A61N 1/378; A61N 1/37223

(57) ABSTRACT

An example medical device includes a battery configured to provide power to the medical device and stimulation circuitry configured to generate an electrical stimulation signal. The medical device includes hibernation control circuitry configured to cause the medical device to enter a hibernation mode in response to a hibernation trigger and exit the hibernation mode in response to a wake-up trigger. The medical device includes a switch configured to open in response to the hibernation control circuitry causing the medical device to enter a hibernation mode and close in response to the hibernation control circuitry causing the medical device to exit the hibernation mode and isolation interface circuitry configured to prevent power leakage from the hibernation control circuitry to the stimulation circuitry when the medical device is in hibernation mode. The stimulation circuitry is not powered by the battery when the medical device is in the hibernation mode.

This application claims priority to U.S. Provisional Application No. 63/215,813, filed Jun. 28, 2021, the entire contents of which is hereby incorporated by reference. This application is related to U.S. Provisional Application No. 63/367,177, filed Jun. 28, 2022, the entire contents of which is hereby incorporated by reference.

### TECHNICAL FIELD

The disclosure relates to medical devices and, more particularly, medical devices that deliver therapy to a patient.

### BACKGROUND

Disease, age, and injury may impair physiological functions of a patient. In some situations, the physiological functions are completely impaired. In other examples, the physiological function may operate sufficiently at some times or under some conditions and operate inadequately at other times or at other conditions. In one example, bladder dysfunction, such as overactive bladder, urgency, or urinary incontinence, is a problem that may afflict people of all ages, genders, and races. Various muscles, nerves, organs and conduits within the pelvic floor cooperate to collect, store and release urine. A variety of disorders may compromise urinary tract performance, and contribute to an overactive bladder, urgency, or urinary incontinence that interferes with normal physiological function. Many of the disorders may be associated with aging, injury or illness.

Urinary incontinence may include urge incontinence and stress incontinence. In some examples, urge incontinence may be caused by disorders of peripheral or central nervous systems that control bladder micturition reflexes. Some patients may also suffer from nerve disorders that prevent proper triggering and operation of the bladder, sphincter muscles or nerve disorders that lead to overactive bladder activities or urge incontinence. In some cases, urinary incontinence may be attributed to improper sphincter function, either in the internal urinary sphincter or external urinary sphincter.

### SUMMARY

In general, the disclosure is directed to devices, systems, and techniques for controlling power delivery to electronics in an implantable medical device (IMD), such as an implantable stimulation device. More particularly, this disclosure is directed to devices, systems, and techniques for implementing a hibernation mode in an IMD. A hibernation mode may significantly reduce the amount of power being used by the IMD.

For many stimulation therapies, the tonic, patterned, pulsed, or cycled stimulation pulses have periods of time where the delivery of stimulation to the body is withheld. During the time periods where therapy is withheld, the majority of the electronics of the IMD may be disconnected from the battery power, or any power source derived from the battery. Such a state may be referred to herein as a hibernation mode. While power is disconnected from the majority of the electronics of the IMD, power may remain connected to a timer and hibernation control circuitry, such that the timer and hibernation control circuitry remain powered and are configured to re-connect power to other circuitry after a predetermined period of time or an external event such as, but not limited to, a magnetic switch closure, energy on the radio antenna or telemetry communication, motion of an accelerometer, the sensing of one or more predetermined physiological parameters of a patient, or the like. This timer and hibernation control circuitry may operate with extremely low power consumption, for example, less than 1 microwatt of power from the battery. Due to the extremely low power consumption, the battery charge may be largely retained while the IMD is not actively providing neurostimulation therapy. This may extend the time period between battery recharges and extend battery life.

For implantable stimulators that are battery powered, the energy drain of the battery is from a combination of the stimulation current delivered to the nervous system and the energy required to operate the supporting electronics. Supporting electronics may include processor circuitry such as a microprocessor, telemetry circuitry, sensors, timing circuitry, and the like. In some cases, the supporting electronics can drain the battery as much as the stimulation current. Hence, by entering the hibernation mode, nearly all of the battery drain may be eliminated when the stimulation therapy is turned off, largely extending the battery life of the stimulator. In some examples, the techniques of this disclosure may practically double the battery life, for example, from 10 years to 20 years.

In one example, the disclosure is directed to a medical device including: a battery configured to provide power to the medical device; stimulation circuitry configured to generate an electrical stimulation signal; hibernation control circuitry configured to cause the medical device to enter a hibernation mode in response to a hibernation trigger and exit the hibernation mode in response to a wake-up trigger; a switch configured to open in response to the hibernation control circuitry causing the medical device to enter a hibernation mode and close in response to the hibernation control circuitry causing the medical device to exit the hibernation mode; and isolation interface circuitry configured to prevent power leakage from the hibernation control circuitry to the stimulation circuitry when the medical device is in hibernation mode, wherein the stimulation circuitry and support circuitry are not powered by the battery when the medical device is in the hibernation mode.

In another example, the disclosure is directed to a method including: determining, by processor circuitry, to enter a hibernation mode; storing, by the processor circuitry and in memory, state and memory information; isolating, by isolation interface circuitry, a boundary between a permanent power domain and a switched power domain; preparing, by the processor circuitry, hibernation control circuitry for hibernation mode; opening, by the hibernation control circuitry, a switch to disconnect power from the switched power domain; and waiting, by the hibernation control circuitry, for a wake-up trigger.

In a further aspect, the disclosure is directed to a method including: receiving, by hibernation control circuitry, a wake-up trigger; closing, by the hibernation control circuitry, a switch to restore power to a switched power domain; reconnecting, by isolation interface circuitry, a boundary between the switched power domain and a permanent power domain; returning, by processor circuitry, the hibernation control circuitry to normal operation; retrieving, by the processor circuitry from memory, state and memory information; and resuming, by the processor circuitry, normal operation of the medical device.

The details of one or more examples are set forth in the accompanying drawings and the description below. Other features, objects, and advantages of the disclosure will be apparent from the description and drawings, and from the claims.

The above summary is not intended to describe each illustrated example or every implementation of the present disclosure.

### BRIEF DESCRIPTION OF DRAWINGS

FIG. 1A is a schematic diagram illustrating an example leadless neurostimulation device.

FIG. 1B is a conceptual diagram illustrating a leg having a leadless neurostimulation device implanted therein near a tibial nerve.

FIG. 1C is a conceptual diagram illustrating an example system that manages delivery of neurostimulation to a patient to manage bladder dysfunction, such as overactive bladder, urgency, or urinary incontinence.

FIG. 2 is a block diagram illustrating an example configuration of an implantable medical devices (IMD).

FIG. 3 is a block diagram illustrating an example configuration of an external programmer.

FIG. 4 is a block diagram illustrating an example neurostimulation device having a hibernation mode according to the techniques of this disclosure.

FIG. 5 is a block diagram illustrating example circuitry of a neurostimulation device according to the techniques of this disclosure.

FIG. 6 is a flow diagram illustrating an example technique for entering a hibernation mode.

FIG. 7 is a flow diagram illustrating an example technique for exiting a hibernation mode.

### DETAILED DESCRIPTION

The disclosure is directed to devices, systems, and techniques for reducing current draw or power drain of an implantable neurostimulation device when the neurostimulation device is not delivering neurostimulation. These techniques may be used with a neurostimulator that may provide therapy for a variety of dysfunctions, diseases or disorders. For purposes of illustration, but without limitation, use of the techniques of this disclosure will be described below with respect to bladder dysfunction. Bladder dysfunction generally refers to a condition of improper functioning of the bladder or urinary tract, and may include, for example, an overactive bladder, urgency, or urinary incontinence. Overactive bladder (OAB) is a patient condition that may include symptoms, such as urgency, with or without urinary incontinence. Urgency is a sudden, compelling urge to urinate, and may often, though not always, be associated with urinary incontinence. Urinary incontinence refers to a condition of involuntary loss of urine, and may include urge incontinence, stress incontinence, or both stress and urge incontinence, which may be referred to as mixed urinary incontinence. As used in this disclosure, the term “urinary incontinence” includes disorders in which urination occurs when not desired, such as stress or urge incontinence. Other bladder dysfunctions may include disorders such as non-obstructive urinary retention.

One type of therapy for treating bladder dysfunction includes delivery of continuous electrical stimulation to a target tissue site within a patient to cause a therapeutic effect during delivery of the electrical stimulation. For example, delivery of electrical stimulation from an implantable medical device (IMD) to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, or branches of any of the aforementioned nerves, may provide an immediate therapeutic effect for bladder dysfunction, such as a desired reduction in frequency of bladder contractions. In some cases, electrical stimulation of the tibial nerve may modulate afferent nerve activities to restore urinary function during the electrical stimulation. However, continuous electrical stimulation (which may include pulsed stimulation) or other types of neurostimulation (e.g., drug delivery therapy) may provide neurostimulation during unnecessary phases of a physiological cycle that may cause undesirable side effects, accommodation, less focused therapy, and increased energy usage by the IMD delivering therapy.

In contrast to this type of continuous neurostimulation therapy, the example devices, systems, and techniques described in this disclosure are directed to managing delivery of neurostimulation therapy in a non-continuous manner which may include on-cycles and off-cycles. For example, an IMD may deliver neurostimulation therapy for a specified period of time followed by a specified period of time when the IMD does not deliver neurostimulation (e.g., withholds delivery of neurostimulation). During periods when the IMD is not delivering neurostimulation, the IMD may enter a hibernation mode to further reduce an amount of power or current draw on a battery powering the IMD. As described herein, a period during which stimulation is delivered (an on-cycle) may include on and off periods (e.g., a duty cycle or bursts of pulses) where even the short inter-pulse durations of time when pulses are not delivered are still considered part of the delivery of stimulation. For example, IMD 16 may not enter the hibernation mode during the short inter-pulse durations of time of an on-cycle when pulses are not delivered. For example, the short inter-pulse duration of time when pulses are not delivered during an on-cycle is still considered part of delivery of stimulation, but during an off-cycle, IMD 16 may be considered as not delivering stimulation.

The disclosure includes discussion of various examples, aspects, and features. Unless otherwise stated, the various examples, aspects, and features are contemplated as being used together in different combinations. For ease of discussion and as a practical matter, each possible combination of features is not expressly recited.

In some examples, a system may be configured to provide stimulation at a neural target located at a distant site from the end organ that is being affected. For example, stimulation sites may be located a relatively large distance from the bladder or bowel, such as a tibial nerve. The system may include of multiple devices (e.g., implantable sensors and implantable stimulation devices) with wireless communication circuitry that allows for wireless communication of information between the devices, which may provide of sensing or therapeutic stimulation. For example, the wireless circuitry may be designed to communicate using Near Field Communication, Bluetooth®, or other wireless protocols.

For tibial stimulation, the techniques of this disclosure may increase the recharge interval from approximately 6 months to one year for the typical patient. In some examples, a clinician may recharge the battery of the IMD during an office visit. In such a case, the IMD may appear to be similar to a primary cell device to the patient. As such, a clinician may utilize a “pay as you go” finance option, where a patient or insurer may pay for a year of therapy during or prior to an annual recharge of the device. While in hibernation mode, an IMD may use less than five microamps of current. In some examples, while in hibernation mode, an IMD may use less one microamp of current. In some examples, the IMD may use on the order of 100 nanoamps of current when in hibernation mode.

For ease of discussion, various examples of are discussed in connection with bladder function. It is to be recognized that bladder function is but one possible application. Various aspects of the present disclosure may also be used in connection with urinary, bowel, and general pelvic floor dysfunction. For the sake of brevity, each type of dysfunction is not repeated for each feature or example discussed herein.

As mentioned above, constant or continuous stimulation may result in undesirable side effects, accommodation, less focused therapy, and increased energy usage by the medical device delivering therapy. Therefore, stimulation may be cycled on and off. When stimulation is cycled off, the neurostimulation device may enter a hibernation mode in which very little current is drawn from the battery powering the neurostimulation device. This may translate into longer recharge intervals and/or longer replacement intervals.

A medical device, such as an IMD may implement the techniques described in this disclosure to deliver stimulation therapy to at least one nerve (e.g., tibial nerve, a spinal nerve, or a pelvic floor nerve) to modulate activity of the nerve via at least one electrode electrically connected to the IMD. The electrical stimulation may be configured to modulate contraction of a detrusor muscle of the patient to cause a decrease in frequency of bladder contractions (to reduce incontinence) or an increase in the frequency of bladder contractions (to promote voiding). Reduction in frequency of bladder contractions may reduce urgency of voiding and may reduce urgency and/or urinary incontinence, and thereby at least partially alleviate bladder dysfunction.

The neurostimulation described herein may be targeted to manage bladder dysfunction, such as an overactive bladder, urgency, urinary incontinence, or even non-obstructive urinary retention. For example, the stimulation may be delivered to target tissue sites normally used to alleviate these types of dysfunction. Although the techniques are primarily described in this disclosure for managing bladder dysfunction, the techniques may also be applied to manage other pelvic floor disorders or disorders relating to other organs, tissues or nerves of the patient. For example, the devices, systems, and techniques described in this disclosure alternatively or additionally may be utilized to manage sexual dysfunction, pelvic pain, fecal urgency or fecal incontinence. Example nerves that may be targeted for therapy include tibial nerves, sacral nerves, pudendal nerves, a dorsal nerve of the penis or clitoris, sural nerves, sciatic nerves, the inferior rectal nerve, and peroneal or perineal nerves. Example organ systems that may be treated for dysfunction may include the large and small bowel, stomach and/or intestines, liver, and spleen, which may be modulated by delivering neurostimulation directly to the organs, to one or nerves innervating the organ, and/or blood supplies reaching the organs. In other examples, therapy may target a spinal cord for pain relief. In other examples, therapy may target a brain for treatment of Parkinson's disease or seizure disorders.

Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination the additional features, such as by repeating every feature each time different examples and uses of the stimulation devices are discussed.

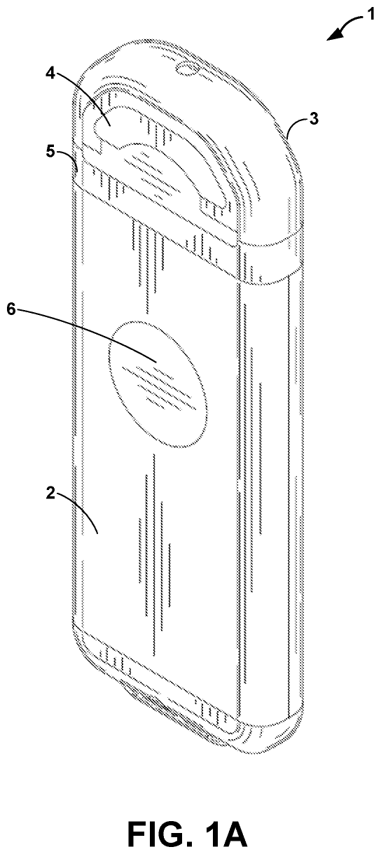

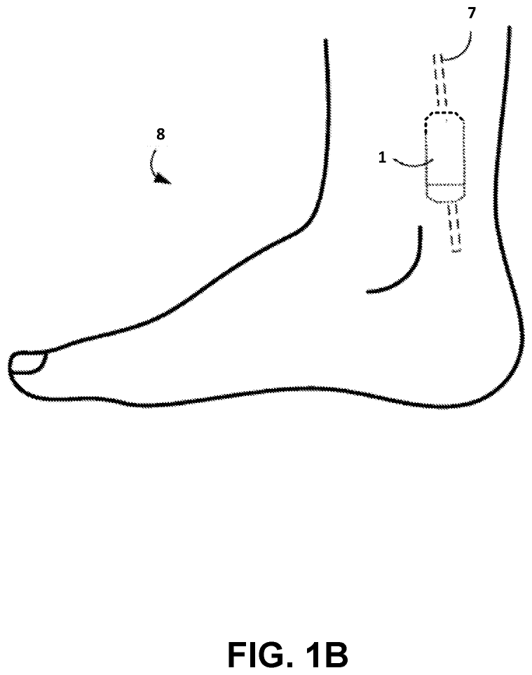

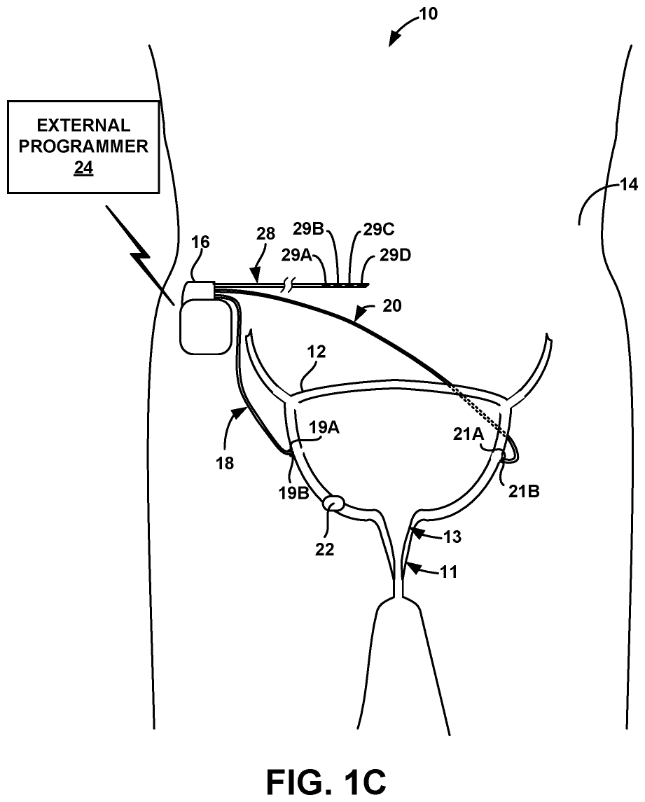

FIG. 1A is a schematic diagram illustrating an example leadless neurostimulation device. Leadless neurostimulation device 1 includes a housing 2 containing components therein configured for delivering neurostimulation therapy, a header unit 3 that includes one or more primary electrodes 4, and a mounting plate 5 that couples housing 2 to header unit 3. Header unit 3 includes at least one primary electrode 4 that forms part of an exterior surface of header unit 3. Housing 2 includes a secondary electrode 6 that forms part of an exterior surface of housing 2 and is positioned on the same side of device 1 as primary electrode 4. In an alternate embodiment not depicted, primary electrode 4 and secondary electrode 6 may be arranged on opposite sides of device 1.

Primary electrode 4 and secondary electrode 6 operate in conjunction with one another to provide stimulation therapy to a target treatment site (e.g., a tibial nerve). Secondary electrode 6 may also be referred to as a case electrode, can electrode or reference electrode. In an example, primary electrode 4 may comprise a cathode and secondary electrode 6 may comprise an anode. In some examples, primary and secondary electrodes 4 and 6 may be characterized as a bipolar pair or system.

The terms “primary” and “secondary” are used to differentiate two or more electrodes that are configured to transmit an electrical signal therebetween. The terms are not used to imply a hierarchy among the electrodes, positive and negative terminal, a total number of electrodes, or a directionality by which a signal is transmitted between the electrodes.

Additional information relating to leadless neurostimulation device 1 may be found in U.S. Patent Publication 2022/0096845A1, the entirety of which is incorporated herein by reference.

FIG. 1B is a conceptual diagram illustrating a leg having a leadless neurostimulation device implanted therein near a tibial nerve. In the example of FIG. 1B, leadless neurostimulation device 1 is implanted near tibial nerve 7 in leg 8 of a patient. For example, leadless neurostimulation device 1 may deliver neurostimulation to the patient to manage bladder dysfunction, such as overactive bladder, urgency, or urinary incontinence. Leadless neurostimulation device 1 may be configured to deliver neurostimulation to tibial nerve 7 in a cycled manner and place leadless neurostimulation device 1 in a hibernation mode when not actively delivering (e.g., withholding) neurostimulation (e.g., an off-cycle).

FIG. 1C is a conceptual diagram illustrating an example system 10 that manages delivery of neurostimulation to patient 14 to manage bladder dysfunction, such as overactive bladder, urgency, or urinary incontinence. As described above, system 10 may be configured to deliver neurostimulation to patient 14 in a cycled manner and place implantable medical device (IMD) 16 in a hibernation mode when not actively delivering (e.g., withholding) neurostimulation (e.g., an off-cycle).

As shown in the example of FIG. 1C, therapy system 10 includes IMD 16 (e.g., an example medical device), which is coupled to leads 18, 20, and 28 and sensor 22. System 10 also includes an external programmer 24, which is configured to communicate with IMD 16 via wireless communication. IMD 16 generally operates as a therapy device that delivers electrical neurostimulation to, for example, a target tissue site proximate a tibial nerve, a spinal nerve, a sacral nerve, a pudendal nerve, dorsal genital nerve, an inferior rectal nerve, a perineal nerve, or other pelvic nerves, or branches of any of the aforementioned nerves. IMD 16 provides electrical stimulation to patient 14 by generating and delivering a programmable electrical stimulation signal (e.g., in the form of electrical pulses or an electrical waveform) to a target a therapy site near lead 28 and, more particularly, near electrodes 29A-29D (collectively referred to as “electrodes 29”) disposed proximate to a distal end of lead 28.

IMD 16 may be surgically implanted in patient 14 at any suitable location within patient 14, such as near the pelvis. In some examples, IMD 16 may be implanted in a subcutaneous location in the side of the lower abdomen or the side of the lower back or upper buttocks. IMD 16 has a biocompatible housing, which may be formed from titanium, stainless steel, a liquid crystal polymer, or the like. The proximal ends of leads 18, 20, and 28 are both electrically and mechanically coupled to IMD 16 either directly or indirectly, e.g., via respective lead extensions. Electrical conductors disposed within the lead bodies of leads 18, 20, and 28 electrically connect sense electrodes (e.g., electrodes 19A, 19B, 21A, and 21B) and stimulation electrodes, such as electrodes 29, to sensing circuitry and a stimulation delivery circuitry (e.g., a stimulation generator) within IMD 16. In the example of FIG. 1C, leads 18 and 20 carry electrodes 19A, 19B (collective referred to as “electrodes 19”) and electrodes 21A, 21B (collectively referred to as “electrodes 21”), respectively. As described in further detail below, electrodes 19 and 21 may be positioned for sensing an impedance of bladder 12, which may increase as the volume of urine within bladder 12 increases. In some examples, system 10 may include electrodes (such as electrodes 19 and 21), a strain gauge, one or more accelerometers, ultrasound sensors, optical sensors, or any other sensor capable of detecting contractions of bladder 12, pressure or volume of bladder 12, or any other indication of the fill cycle of bladder 12 and/or possible bladder dysfunctional states.

In other examples, system 10 may use sensors other than electrodes 19 and 21 for sensing bladder volume, or not use any sensors at all. For example, external programmer 24 may receive user input identifying a voiding event, perceived level of fullness, or the like. The user input may be in the form of a voiding journal analyzed by external programmer 24 or IMD 16 or individual user inputs associated with respective voiding events, leakage, or any other event related to a phase of the physiological cycle. External programmer 24 and/or IMD 16 may use this user input to generate estimated fill cycles and determine when to exit hibernation mode and deliver stimulation and when to withhold stimulation and enter hibernation mode. In other words, one or more physiological markers may be identified from user input and utilized for determining when to enter and exit hibernation mode. The user input may be in addition to or instead of sensors such as electrodes 19A and 21A for detecting a physiological marker.

One or more medical leads, e.g., leads 18, 20, and 28, may be connected to IMD 16 and surgically or percutaneously tunneled to place one or more electrodes carried by a distal end of the respective lead at a desired nerve or muscle site, e.g., one of the previously listed target therapy sites such as a tissue site proximate a tibial, spinal, sacral, or pudendal nerve. For example, lead 28 may be positioned such that electrodes 29 deliver electrical stimulation to a tibial, spinal, sacral, or pudendal nerve to reduce a frequency and/or magnitude of contractions of bladder 12. Additional electrodes of lead 28 and/or electrodes of another lead may provide additional stimulation therapy to other nerves or tissues as well. In FIG. 1C, leads 18 and 20 are placed proximate to an exterior surface of the wall of bladder 12 at first and second locations, respectively. In other examples of therapy system 10, IMD 16 may be coupled to more than one lead that includes electrodes for delivery of electrical stimulation to different stimulation sites within patient 14, e.g., to target different nerves.

In the example shown in FIG. 1C, leads 18, 20, 28 are cylindrical. Electrodes 19, 20, 29 of leads 18, 20, 28, respectively, may be ring electrodes, segmented electrodes, partial ring electrodes or any suitable electrode configuration. Segmented and partial ring electrodes each extend along an arc less than 360 degrees (e.g., 90-120 degrees) around the outer perimeter of the respective lead 18, 20, 28. In some examples, segmented electrodes 29 of lead 28 may be useful for targeting different fibers of the same or different nerves to generate different physiological effects (e.g., therapeutic effects). In examples, one or more of leads 18, 20, 28 may be, at least in part, paddle-shaped (e.g., a “paddle” lead), and may include an array of electrodes on a common surface, which may or may not be substantially flat.

In some examples, one or more of electrodes 19, 20, 29 may be cuff electrodes that are configured to extend at least partially around a nerve (e.g., extend axially around an outer surface of a nerve). Delivering electrical stimulation via one or more cuff electrodes and/or segmented electrodes may help achieve a more uniform electrical field or activation field distribution relative to the nerve, which may help minimize discomfort to patient 14 that results from the delivery of electrical stimulation. An electrical field may define the volume of tissue that is affected when the electrodes 19, 20, 29 are activated. An activation field represents the neurons that will be activated by the electrical field in the neural tissue proximate to the activated electrodes.

The illustrated numbers and configurations of leads 18, 20, and 28 and electrodes carried by leads 18, 20, and 28 are merely exemplary. Other configurations, e.g., numbers and positions of leads and electrodes are also contemplated. For example, in other implementations, IMD 16 may be coupled to additional leads or lead segments having one or more electrodes positioned at different locations proximate the spinal cord or in the pelvic region of patient 14. The additional leads may be used for delivering different stimulation therapies or other electrical stimulations to respective stimulation sites within patient 14 or for monitoring at least one physiological marker of patient 14.

In accordance with some examples of the disclosure, IMD 16 delivers electrical stimulation based on a stimulation program to at least one of a tibial nerve, spinal nerve (e.g., a sacral nerve), a pudendal nerve, dorsal genital nerve, an inferior rectal nerve, or a perineal nerve to provide a therapeutic effect that reduces or eliminates a dysfunctional state such as overactive bladder. The desired therapeutic effect may be an inhibitory physiological response related to voiding of patient 14, such as a reduction in bladder contraction frequency by a desired level or degree (e.g., percentage).

The stimulation program may define various parameters of the stimulation waveform and electrode configuration which result in a predetermined stimulation intensity being delivered to the targeted nerve or tissue. In some examples, the stimulation program defines parameters for at least one of a current or voltage amplitude of the stimulation signal, a frequency or pulse rate of the stimulation, the shape of the stimulation waveform, a duty cycle of the stimulation, a pulse width of the stimulation, and/or the combination of electrodes 29 and respective polarities of the subset of electrodes 29 used to deliver the stimulation. Together, these stimulation parameter values may be used to define the stimulation intensity (also referred to herein as a stimulation intensity level). In some examples, if stimulation pulses are delivered in bursts, a burst duty cycle also may contribute to stimulation intensity. Also, independent of intensity, a particular pulse width and/or pulse rate may be selected from a range suitable for causing the desired therapeutic effect after stimulation is terminated and, optionally, during stimulation. In addition, as described herein, a period during which stimulation is delivered may include on and off periods (e.g., a duty cycle or bursts of pulses) where even the short inter-pulse durations of time when pulses are not delivered are still considered part of the delivery of stimulation. For example, IMD 16 may not enter the hibernation mode during the short inter-pulse durations of time when pulses are not delivered. For example, the short inter-pulse duration of time when pulses are not delivered during an on-cycle is still considered part of delivery of stimulation, but during an off-cycle, IMD 16 may be considered as not delivering stimulation.

The stimulation programs may also define a period during which IMD 16 delivers stimulation (e.g., on-cycle) and a period during which IMD 16 withholds stimulation delivery (e.g., off-cycle). The period during which system 10 withholds stimulation delivery is a period in which no stimulation program is active for IMD 16 (e.g., IMD 16 is not tracking pulse durations or inter-pulse durations that occur as part of the electrical stimulation delivery scheme). During such times, IMD 16 may enter the hibernation mode. Typically, a period during which system 10 withholds neurostimulation is on the order of weeks, days, minutes or hours, not tenths of a second or several seconds.

These periods may be programmed to be absolute or linked to triggers, such as sensor signals or telemetry circuitry signals. In some examples, IMD 16 may be configured to deliver different types of stimulation therapy at different times during a physiological cycle of patient 14. For example, IMD 16 may deliver stimulation configured to reduce or eliminate bladder contractions to promote urine retention and/or increased bladder capacity and then deliver stimulation configured to promote urination (e.g., increased frequency or magnitude of bladder contractions) for a user requested voiding event or once a voiding event is detected to have begun.

In some examples, the hibernation includes a plurality of hibernation periods in a sequence, with brief periods of time out of hibernation mode. For example, IMD 16 may come out of hibernation mode to run diagnostic tests or advertise for telemetry, before reentering hibernation mode if there is not an issue with the results of the diagnostic tests or a request to connect to an external device. In some examples, the hibernation time can be varied by algorithms that monitor therapy efficacy (e.g., using implanted or external sensors) and adjust the therapy stimulation dose.

System 10 may also include an external programmer 24, as shown in FIG. 1C. External programmer 24 may be a clinician programmer or patient programmer. In some examples, external programmer 24 may be a wearable communication device, with a therapy request input integrated into a key fob or a wristwatch, handheld computing device, smart phone, computer workstation, or networked computing device. External programmer 24 may include a user interface that is configured to receive input from a user (e.g., patient 14, a patient caretaker, or a clinician). In some examples, the user interface includes, for example, a keypad and a display, which may for example, be a liquid crystal display (LCD) or light emitting diode (LED) display. The keypad may take the form of an alphanumeric keypad or a reduced set of keys associated with particular functions. External programmer 24 may additionally or alternatively include a peripheral pointing device, such as a mouse, via which a user may interact with the user interface. In some examples, a display of external programmer 24 may include a touch screen display, and a user may interact with external programmer 24 via the display. It should be noted that the user may also interact with external programmer 24 and/or IMD 16 remotely via a networked computing device. In some examples, external programmer 24 may be configured to interoperate with leadless neurostimulation device 1.

A user, such as a physician, technician, surgeon, electrophysiologist, or other clinician, may also interact with external programmer 24 or another separate programmer (not shown), such as a clinician programmer, to communicate with IMD 16. Such a user may interact with a programmer to retrieve physiological or diagnostic information from IMD 16. The user may also interact with a programmer to program IMD 16, e.g., select values for the stimulation parameter values with which IMD 16 generates and delivers stimulation and/or the other operational parameters of IMD 16, such as magnitudes of stimulation energy, user requested periods for stimulation or periods to prevent stimulation, or any other such user customization of therapy. As discussed herein, the user may also provide input to external programmer 24 indicative of physiological events such as bladder fill level perception and void events.

For example, the user may use a programmer to retrieve information from IMD 16 regarding the contraction frequency of bladder 12 and/or voiding events. As another example, the user may use a programmer to retrieve information from IMD 16 regarding the performance or integrity of IMD 16 or other components of system 10, such as leads 18, 20, and 28, or a power source of IMD 16. In some examples, this information may be presented to the user as an alert if a system condition that may affect the efficacy of therapy is detected.

Patient 14 may, for example, use a keypad or touch screen of external programmer 24 to request IMD 16 to deliver or terminate the electrical stimulation, such as when patient 14 senses that a leaking episode may be imminent or when an upcoming void may benefit from terminating therapy that promotes urine retention. In this way, patient 14 may use external programmer 24 to provide a therapy request to control the delivery of the electrical stimulation “on demand,” e.g., when patient 14 deems the stimulation therapy desirable. In the case where patient 14 uses external programmer 24 to request IMD 16 to deliver therapy, IMD 16 may exit the hibernation mode in order to deliver the electrical stimulation. In some examples, patient 14 may use external programmer 24 to request IMD 16 to terminate electrical stimulation. In the case where patient 14 uses external programmer 24 to terminate electrical stimulation, IMD 16 may enter hibernation mode. Patient 14 may also use external programmer 24 to provide other information to IMD 16, such as information indicative of a phase of a physiological cycle, such as the occurrence of a voiding event.

External programmer 24 may provide a notification to patient 14 when the electrical stimulation is being delivered or notify patient 14 of the prospective termination of the electrical stimulation. In addition, notification of termination may be helpful so that patient 14 knows that a voiding event may be more probable and/or the end of the fill cycle is nearing such that the bladder should be emptied (e.g., patient 14 should visit a restroom). In such examples, external programmer 24 may display a visible message, emit an audible alert signal or provide a somatosensory alert (e.g., by causing a housing of external programmer 24 to vibrate). In other examples, the notification may indicate when therapy is available (e.g., a countdown in minutes, or indication that therapy is ready) during the physiological cycle. In this manner, external programmer 24 may wait for input from patient 14 prior to terminating the electrical stimulation that reduces bladder contraction or otherwise promotes urine retention. Patient 14 may enter input that either confirms termination of the electrical stimulation so that the therapy stops for voiding purposes, confirms that the system should maintain therapy delivery until patient 14 may void, and/or confirms that patient 14 is ready for another different stimulation therapy that promotes voiding during the voiding event.

In the event that no input is received within a particular range of time when a voiding event is predicted, external programmer 24 may wirelessly transmit a signal that indicates the absence of patient input to IMD 16. IMD 16 may then elect to continue stimulation until the patient input is received, or terminate stimulation to avoid tissue damage, based on the programming of IMD 16.

IMD 16 and external programmer 24 may communicate via wireless communication using any techniques known in the art. Examples of communication techniques may include, for example, low frequency, radiofrequency (RF) telemetry, or inductive coupling, but other techniques are also contemplated. In some examples, external programmer 24 may include a programming lead that may be placed proximate to the body of patient 14 near the IMD 16 implant site in order to improve the quality or security of communication between IMD 16 and external programmer 24.

In one example described herein, a medical device (e.g., IMD 16) includes a battery configured to provide power to the medical device. The medical device also includes stimulation circuitry configured to generate an electrical stimulation signal. The medical device includes hibernation control circuitry configured to cause the medical device to enter a hibernation mode in response to a hibernation trigger and exit the hibernation mode in response to a wake-up trigger. The medical device includes a switch configured to open in response to the hibernation control circuitry causing the medical device to enter a hibernation mode and close in response to the hibernation control circuitry causing the medical device to exit the hibernation mode. For example, the switch opening disconnects the battery from certain circuitry of IMD 16 and the switch closing reconnects the battery from the certain circuitry of IMD 16. The medical device also includes isolation interface circuitry configured to prevent power leakage from the hibernation control circuitry to the stimulation circuitry when the medical device is in hibernation mode. In this example, the stimulation circuitry is not powered by the battery when the medical device is in the hibernation mode.

In some examples, system 10 is configured to detect voiding events and, responsive to the detection, control IMD 16 to terminate delivery of the neuro stimulation therapy and enter hibernation mode.

In some examples, sensor 22 may sense a phase of a physiological cycle. For example, the sensor may be a pressure sensor that may sense a phase of a bladder fill cycle and the medical device may exit the hibernation mode after sensing a predetermined bladder pressure indicative of the bladder becoming fuller.

The magnitude of the fill level may be a physiological marker for the bladder fill cycle. In one example, system 10 may detect the magnitude of the fill level by detecting a pressure level of bladder 14 (e.g., via sensor 22). For example, one or more pressure or stretch sensors may be attached to the exterior of bladder 14 or implanted within the bladder. As another example, system 10 may detect the magnitude of the fill level by detecting an impedance level of bladder 14, such as by monitoring the impedance between electrodes 19 and 21 of FIG. 1C.

IMD 16 may detect a contraction of bladder 12 using any suitable technique, such as based on a sensed one or more physiological parameters that may be a physiological marker for the physiological cycle. In one example, a physiological marker is an impedance of bladder 12. In the example shown in FIG. 1C, IMD 16 may determine impedance of bladder 12 using a four-wire (or Kelvin) measurement technique. In other examples, IMD 16 may measure bladder impedance using a two-wire sensing arrangement. In either case, IMD 16 may transmit an electrical measurement signal, such as a current, through bladder 12 via leads 18 and 20, and determine impedance of bladder 12 based on the transmitted electrical signal. Such an impedance measurement may be utilized to determine a fullness of bladder 12, or the like.

In the example four-wire arrangement shown in FIG. 1C, electrodes 19A and 21A and electrodes 19B and 21B, may be located substantially opposite each other relative to the center of bladder 12. For example, electrodes 19A and 21A may be placed on opposing sides of bladder 12, either anterior and posterior or left and right. In FIG. 1C, electrodes 19 and 21 are shown placed proximate to an exterior surface of the wall of bladder 12. In some examples, electrodes 19 and 21 may be sutured or otherwise affixed to the bladder wall. In other examples, electrodes 19 and 21 may be implanted within the bladder wall. To measure the impedance of bladder 12, IMD 16 may source an electrical signal, such as current, to electrode 19A via lead 18, while electrode 21A via lead 20 sinks the electrical signal. IMD 16 may then determine the voltage between electrode 19B and electrode 21B via leads 18 and 20, respectively. IMD 16 determines the impedance of bladder 12 using a known value of the electrical signal sourced the determined voltage.

In other examples, electrodes 19 and 21 may be used to detect an EMG of the detrusor muscle. This EMG may be used to determine the frequency of bladder contractions and the physiological marker of patient 14. The EMG may also be used to detect the strength of the bladder contractions in some examples. As an alternative, or in addition, to an EMG, a strain gauge or other device may be used to detect the status of bladder 12, e.g., by sensing forces indicative of bladder contractions.

In the example of FIG. 1C, IMD 16 also includes a sensor 22 for detecting changes in the contraction of bladder 12. Sensor 22 may include, for example, a pressure sensor for detecting changes in bladder pressure, electrodes for sensing pudendal or sacral afferent nerve signals, electrodes for sensing urinary sphincter EMG signals (or anal sphincter EMG signals in examples in which system 10 provides therapy to manage fecal urgency or fecal incontinence), or any combination thereof. In examples in which sensor 22 is a pressure sensor or a stretch sensor, sensor 22 may be a remote sensor that wirelessly transmits signals to IMD 16 or may be carried on one of leads 18, 20, or 28 or an additional lead coupled to IMD 16. In some examples, IMD 16 may determine whether a contraction frequency of bladder 12 has occurred based on a pressure signal generated by sensor 22.

In examples in which sensor 22 includes one or more electrodes for sensing afferent nerve signals, the sense electrodes may be carried on one of leads 18, 20, or 28 or an additional lead coupled to IMD 16. In examples in which sensor 22 includes one or more sense electrodes for generating a urinary sphincter EMG, the sense electrodes may be carried on one of leads 18, 20, or 28 or additional leads coupled to IMD 16. In any case, in some examples, IMD 16 may control the timing of the delivery of the electrical stimulation based on input received from sensor 22.

Sensor 22 may comprise a patient motion sensor, such as an accelerometer, that generates a signal indicative of patient activity level or posture state. In some examples, IMD 16 may terminate or resume the delivery of the electrical stimulation to patient 14 upon detecting a patient activity level falling below or exceeding a particular threshold based on the signal from the motion sensor. For example, if the patient activity level that is greater than or equal to a threshold (which may be stored in a memory of IMD 16) may indicate that there is an increase in the probability that an involuntary voiding event will occur, and, therefore, system 10 should exit the hibernation mode and begin delivering electrical stimulation. In other examples, IMD 16 may use sensor 22 to identify posture states known to require the desired therapeutic effect. For example, patient 14 may be more prone to an involuntary voiding event when patient 14 is in an upright posture state compared to a lying down posture state. In any event, electrodes 19 and 21 and sensor 22 may be configured to detect voiding events and/or the magnitude of a fill level of bladder 12 during the fill cycle. Any of these detected features from patient 14 may be used as a trigger to IMD 16 to enter or exit the hibernation mode.

As discussed above, system may monitor the fill cycle of bladder 12 by detecting subsequent voiding events over time. In some examples, system 10 may detect voiding events by receiving an indication of a user input (e.g., via external programmer 24) representative of an occurrence of a voiding event. In other words, external programmer 24 may receive input from the user identifying that a voiding event occurred, the beginning of a voiding event, and/or the end of the voiding event. In other examples, system 10 may automatically detect voiding events without receiving user input via external programmer 24. System 10 may instead detect voiding events by detecting at least one of a pressure of the bladder, a flow of urine from the bladder, a wetness of an article external to patient 14, a volume of the bladder, an electromyogram (EMG) signal, a nerve recording, a posture change, a physical location of patient 14 within a structure such as a house or care facility, or a toilet use event. Some sensors external to patient 14 may communicate with external programmer 24 and/or IMD 16 to provide this information indicative of likely voiding events. For example, wetness may be detected by a moisture sensor (e.g., electrical impedance or chemical sensor) embedded in an undergarment worn by patient 14 and transmitted to IMD 16 or external programmer 24. Similarly, a toilet may include a presence sensor that detects when patient 14 is using the toilet (e.g., an infrared sensor, thermal sensor, or pressure senor) and transmits a signal indicating the presence of patient 14 to IMD 16 or external programmer 24. In this manner, non-invasively obtained data may provide information indicative of voiding events without implanted sensors, and such information may be used to determine when to enter or exit the hibernation mode.

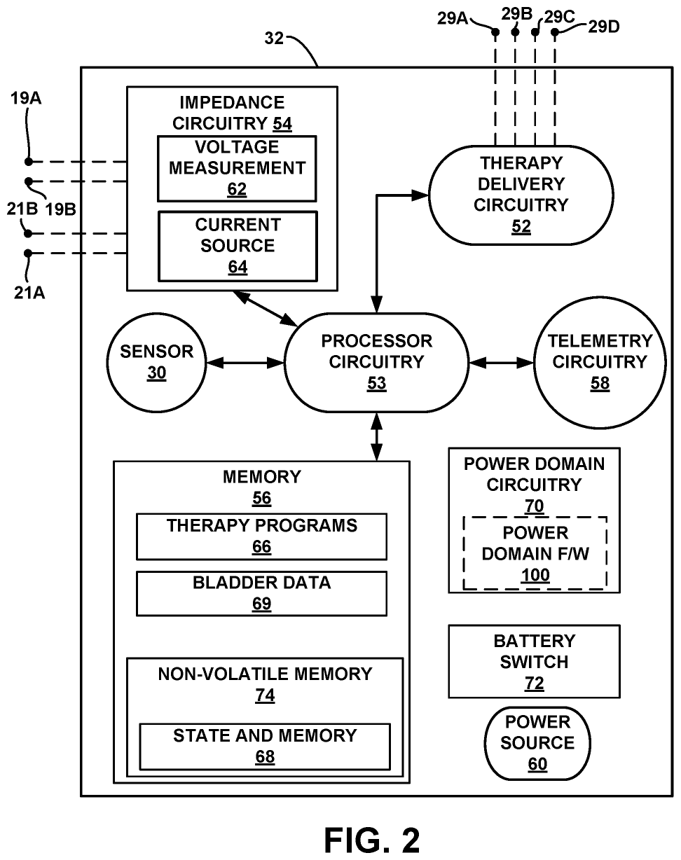

FIG. 2 is a block diagram illustrating an example configuration of an IMD. As shown in FIG. 2, IMD 32, which may be an example of leadless neurostimulation device 1 or IMD 32, includes sensor 22, processor circuitry 53, stimulation circuitry 52, impedance circuitry 54, memory 56, telemetry circuitry 58, hibernation control circuitry 70, isolation interface circuitry 72, and power source 60. In other examples, IMD 32 may include a greater or fewer number of components. For example, in the case where IMD 32 represents leadless neurostimulation device 1, IMD 32 does not include leads, but rather electrodes are disposed on the surface of IMD 32.

In general, IMD 32 may comprise any suitable arrangement of hardware, alone or in combination with software and/or firmware, to perform the techniques attributed to IMD 32 and components of IMD 32. In various examples, IMD 32 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 32 also, in various examples, may include a memory 56, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although processor circuitry 53, stimulation circuitry 52, impedance circuitry 54, and telemetry circuitry 58 are described as separate circuitry, in some examples, processor circuitry 53, stimulation circuitry 52, impedance circuitry 54, and telemetry circuitry 58 are functionally integrated. In some examples, processor circuitry 53, stimulation circuitry 52, impedance circuitry 54, and telemetry circuitry 58 correspond to individual hardware units, such as microprocessors, ASICs, DSPs, FPGAs, or other hardware units. In further examples, any of processor circuitry 53, stimulation circuitry 52, impedance circuitry 54, and telemetry circuitry 58 may correspond to multiple individual hardware units such as microprocessors, ASICs, DSPs, FPGAs, or other hardware units.

Memory 56 stores therapy programs 66 that specify stimulation parameter values and electrode combinations for the electrical stimulation provided by IMD 32. Therapy programs 66 may also store information regarding determining and using physiological parameters, information regarding physiological cycles and/or dysfunctional states, or any other information required by IMD 32 to deliver stimulation therapy based on one or more physiological parameters of patient 14. In some examples, memory 56 also stores bladder data 69, which processor circuitry 53 may use for controlling the timing of the delivery of the electrical stimulation (e.g., phases of physiological cycles that define when to deliver and withhold stimulation). For example, bladder data 69 may include threshold values or baseline values for at least one of bladder impedance, bladder pressure, afferent nerve signals, bladder contraction frequency, or external urinary sphincter EMG templates for use as physiological markers for an associated physiological cycle. Bladder data 69 may also include timing information and physiological markers associated with physiological events, such as a voiding event.

Memory 56 may also store state and memory information 68. For example, when IMD 32 is about to enter the hibernation mode, processor circuitry 53 may store certain state and memory information in state and memory information 68. State and memory information 68 may then be accessed by processor circuitry 53 when IMD 32 is exiting the hibernation mode, which processor circuitry may use to restore IMD 32 to a normal operative state. State and memory information may include time information indicative of when the hibernation mode is being entered, a desired exit time, length of time of last therapy delivery, or the like. State and memory information may also include information regarding sensor acquisition and therapy sequences. State and memory information may include algorithm states and data for assessments that are underway, such as bladder filling calculations or therapy titration calculations. State and memory information may also include diagnostic data for the device state, such as battery voltage, electrode impedances, and/or telemetry logs.

Information related to sensed bladder contractions, bladder impedance and/or posture of patient 14 may be stored in bladder data 69. Bladder data 69 may be retrieved by a user, and/or used by processor circuitry 53 for adjustment of stimulation parameters (e.g., amplitude, pulse width, and pulse rate). In some examples, memory 56 includes separate memories for storing instructions, electrical signal information, stimulation programs 66, state and memory information 68, and bladder data 69. In some examples, processor circuitry 53 selects new stimulation parameters for a therapy program 66 or new stimulation program from stimulation programs 66 to use in the delivery of the electrical stimulation based on patient input or sensor signals. In some examples, processor circuitry 53 may use bladder data 69 to determine efficacy of a therapy program and may adjust periods of stimulation and periods of withholding of stimulation and the associated operational mode and hibernation mode based on the determined efficacy of the therapy program.

Generally, stimulation circuitry 52 generates and delivers electrical stimulation under the control of processor circuitry 53. As used herein, controlling the delivery of electrical stimulation may also include controlling the termination of stimulation to achieve the different stimulation and non-stimulation phases. In some examples, processor circuitry 53 controls stimulation circuitry 52 by accessing memory 56 to selectively access and load at least one of stimulation programs 66 to stimulation circuitry 52. For example, in operation, processor circuitry 53 may access memory 56 to load one of stimulation programs 66 to stimulation circuitry 52. In other examples, stimulation circuitry 52 may access memory 56 and load one of the stimulation programs 66.

By way of example, processor circuitry 53 may access memory 56 to load one of stimulation programs 66 to stimulation circuitry 52 for delivering the electrical stimulation to patient 14. A clinician or patient 14 may select a particular one of stimulation programs 66 from a list using a programming device, such as external programmer 24 or a clinician programmer. Processor circuitry 53 may receive the selection via telemetry circuitry 58. Stimulation circuitry 52 delivers the electrical stimulation to patient 14 according to the selected program for an extended period of time, such as minutes, hours, days, weeks, or until patient 14 or a clinician manually stops or changes the program.

Therapy delivery circuitry 52 delivers electrical stimulation according to stimulation parameters. In some examples, stimulation circuitry 52 delivers electrical stimulation in the form of electrical pulses. In such examples, relevant stimulation parameters may include a voltage amplitude, a current amplitude, a pulse rate, a pulse width, a duty cycle, or the combination of electrodes 29 that stimulation circuitry 52 uses to deliver the stimulation signal. In other examples, stimulation circuitry 52 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes 29 stimulation circuitry 52 uses to deliver the stimulation signal.

In some examples, the stimulation parameters for the stimulation programs 66 may be selected to relax bladder 12, e.g., to reduce a frequency of contractions of bladder 12, after termination of the electrical stimulation. An example range of stimulation parameters for the electrical stimulation that are likely to be effective in treating bladder dysfunction, e.g., upon application to the tibial, spinal, sacral, pudendal, dorsal genital, inferior rectal, or perineal nerves, are as follows:

1. Frequency or pulse rate: between about 0.5 Hz and about 500 Hz, such as between about 1 Hz and about 250 Hz, between about 1 Hz and about 20 Hz, or about 10 Hz.

2. Amplitude: between about 0.1 volts and about 50 volts, such as between about 0.5 volts and about 20 volts, or between about 1 volt and about 10 volts. Alternatively, the amplitude may be between about 0.1 milliamps (mA) and about 50 mA, such as between about 0.5 mA and about 20 mA, or between about 1 mA and about 10 mA.

3. Pulse Width: between about 10 microseconds (μs) and about 5000 μs, such as between about 100 μs and about 1000 μs, or between about 100 μs and about 200 μs.

When IMD 32 is monitoring the fill level of the bladder to determine the status of the bladder fill cycle, processor circuitry 53 may monitor impedance of bladder 12 for a predetermined duration of time to detect contractions of bladder 12, and determine the baseline contraction frequency of bladder 12 by determining a number of contractions of bladder 12 in the predetermined duration of time. In other examples, electrodes 19 or 21 may be used to detect an EMG of the detrusor muscle to identify bladder contraction frequencies. Alternatively, a strain gauge sensor signal output or other measure of bladder contraction change may be used to detect the physiological marker of bladder 12. Each of these alternative methods of monitoring the fill level and/or voiding event of bladder 12 may be used in some examples.

In the example illustrated in FIG. 2, impedance circuitry 54 includes voltage measurement circuitry 62 and current source 64, and may include an oscillator (not shown) or the like for producing an alternating signal. In some examples, as described above with respect to FIG. 1, impedance circuitry 54 may use a four-wire, or Kelvin, arrangement. As an example, processor circuitry 53 may periodically control current source 64 to, for example, source an electrical current signal through electrode 19A and sink the electrical current signal through electrode 21A. In some examples, for collection of impedance measurements, current source 64 may deliver electrical current signals that do not deliver stimulation therapy to bladder 12, e.g., sub-threshold signals, due to, for example, the amplitudes or widths of such signals and/or the timing of delivery of such signals. Impedance circuitry 54 may also include a switching circuitry (not shown) for selectively coupling electrodes 19A, 19B, 21A, and 21B to current source 64 and voltage measurement circuitry 62. Voltage measurement circuitry 62 may measure the voltage between electrodes 19B and 21B. Voltage measurement circuitry 62 may include sample and hold circuitry or other suitable circuitry for measuring voltage amplitudes. Processor circuitry 53 determines an impedance value from the measure voltage values received from voltage measurement circuitry 52.

In other examples, processor circuitry 53 may monitor signals received from sensor 22 to detect contraction of bladder 12 and determine the baseline contraction frequency. In some examples, sensor 22 may be a pressure sensor for detecting changes in pressure of bladder 12, which processor circuitry 53 may correlate to contractions of bladder 12. Processor circuitry 53 may determine a pressure value based on signals received from sensor 22 and compare the determined pressure value to a threshold value stored in bladder data 69 to determine whether the signal is indicative of a contraction of bladder 12. In some implementations, processor circuitry 53 monitors pressure of bladder 12 to detect contractions of bladder 12 for a predetermined duration of time, and determines a contraction frequency of bladder 12 by calculating a number of contractions of bladder 12 in the predetermined time period.

In some examples, processor circuitry 53 may cause contraction frequency information to be stored as bladder data 69 in memory 56, and may utilize the changes to contraction frequency to track the fill level of the bladder fill cycle or otherwise track the phase of the fill cycle. In some implementations, processor circuitry 53 may, automatically or under control of a user, determine the contraction frequency over the fill cycle. Processor circuitry 53 may determine that an increase in contraction frequency indicates a later phase of the fill cycle. In some examples, processor circuitry 53 may track bladder contractions using EMG signals of patient 14. In some implementations, sensor 22 may include an EMG sensor, and processor circuitry 53 may generate an EMG from the received signals generated by sensor 22. Sensor 22 may be implanted proximate to a muscle which is active when bladder 12 is contracting, such as a detrusor muscle. Processor circuitry 53 may compare an EMG collected during the second time period to EMG templates stored as bladder data 69 (e.g., a short-term running average) to determine whether the contractions of bladder 12 are indicative of particular phases of the bladder fill cycle.

In other examples, sensor 22 may be a pressure sensor and processor circuitry 53 may monitor signals received from sensor 22 during at least a portion of the second time period to detect contraction of bladder 12. In some examples, processor circuitry 53 substantially continuously monitors pressure of bladder 12, at least during the second time periods, to detect contraction of bladder 12, and determines a contraction frequency of bladder 12 by determining a number of contractions of bladder 12 in a specified time period. Sensor 22 may also provide longer-term changes in pressure to track the bladder fill status (e.g., increased bladder volume may correspond to increased bladder pressure).

In the example of FIG. 2, stimulation circuitry 52 drives electrodes on a single lead 28. Specifically, stimulation circuitry 52 delivers electrical stimulation to tissue of patient 14 via selected electrodes 29A-29D carried by lead 28. A proximal end of lead 28 extends from the housing of IMD 32 and a distal end of lead 28 extends to a target therapy site, such as a tibial nerve, a spinal nerve (e.g., an S3 nerve), or a therapy site within the pelvic floor, such as tissue sites proximate a sacral nerve, a pudendal nerve, a dorsal genital nerve, an inferior rectal nerve, a perineal nerve, a hypogastric nerve, a urinary sphincter, or any combination thereof. In other examples, stimulation circuitry 52 may deliver electrical stimulation with electrodes on more than one lead and each of the leads may carry one or more electrodes. The leads may be configured as an axial lead with ring electrodes or segmented electrodes and/or paddle leads with electrode pads arranged in a two-dimensional array. The electrodes may operate in a bipolar or multi-polar configuration with other electrodes, or may operate in a unipolar configuration referenced to an electrode carried by the device housing or “can” of IMD 32.

As previously described, sensor 22 may comprise a pressure sensor configured to detect changes in bladder pressure, electrodes for sensing pudendal or sacral afferent nerve signals, or electrodes for sensing external urinary sphincter EMG signals (or anal sphincter signals in examples in which IMD 32 provides fecal urgency or fecal incontinence therapy), or any combination thereof. Additionally, or alternatively, sensor 22 may comprise a motion sensor, such as a two-axis accelerometer, three-axis accelerometer, one or more gyroscopes, pressure transducers, piezoelectric crystals, or other sensors that generate a signal that changes as patient activity level or posture state changes. Processor circuitry 53 may detect a physiological marker indicative of point during a bladder fill cycle. Sensor 22 may also be a motion sensor that is responsive to tapping (e.g., by patient 14) on skin superior to IMD 32. Processor circuitry 53 may be configured to log patient input using this tapping method (e.g., tapping may indicate that a voiding event is occurring). Alternatively, or in addition, processor circuitry 53 may control therapy circuitry 52 to deliver or terminate electrical stimulation delivery in response to the tapping or certain pattern of tapping.

In examples in which sensor 22 includes a motion sensor, processor circuitry 53 may determine a patient activity level or posture state based on a signal generated by sensor 22. This patient activity level may be, for example, sitting, exercising, working, running, walking, or any other activity of patient 14. For example, processor circuitry 53 may determine a patient activity level by sampling the signal from sensor 22 and determining a number of activity counts during a sample period, where each activity level of a plurality of activity levels is associated with respective activity counts. In one example, processor circuitry 53 compares the signal generated by sensor 22 to one or more amplitude thresholds stored within memory 56, and identifies each threshold crossing as an activity count. The physical activity may be indicative of a fill level, a voiding event, or any other physiological marker related to the bladder fill cycle.

In some examples, processor circuitry 53 may control stimulation circuitry 52 to deliver or terminate the electrical stimulation and exit or enter the hibernation mode based on patient input received via telemetry circuitry 58. Telemetry circuitry 58 includes any suitable hardware, firmware, software or any combination thereof for communicating with another device, such as external programmer 24 (FIG. 1). Under the control of processor circuitry 53, telemetry circuitry 58 may receive downlink telemetry, e.g., patient input, from and send uplink telemetry, e.g., an alert, to external programmer 24 with the aid of an antenna, which may be internal and/or external. Processor circuitry 53 may provide the data to be uplinked to external programmer 24 and the control signals for the telemetry circuit within telemetry circuitry 58, and receive data from telemetry circuitry 58.

Generally, processor circuitry 53 may control telemetry circuitry 58 to exchange information with external programmer 24 and/or another device external to IMD 32. Processor circuitry 53 may transmit operational information and receive stimulation programs or stimulation parameter adjustments via telemetry circuitry 58. Also, in some examples, IMD 32 may communicate with other implanted devices, such as stimulators, control devices, or sensors, via telemetry circuitry 58.

Power source 60 delivers operating power to the components of IMD 32. Power source 60 may include a battery and a power generation circuit to produce the operating power. In some examples, the battery may be rechargeable to allow extended operation. Recharging may be accomplished through proximal inductive interaction between an external charger and an inductive charging coil within IMD 32. In other examples, an external inductive power supply may transcutaneously power IMD 32 whenever electrical stimulation is to occur.

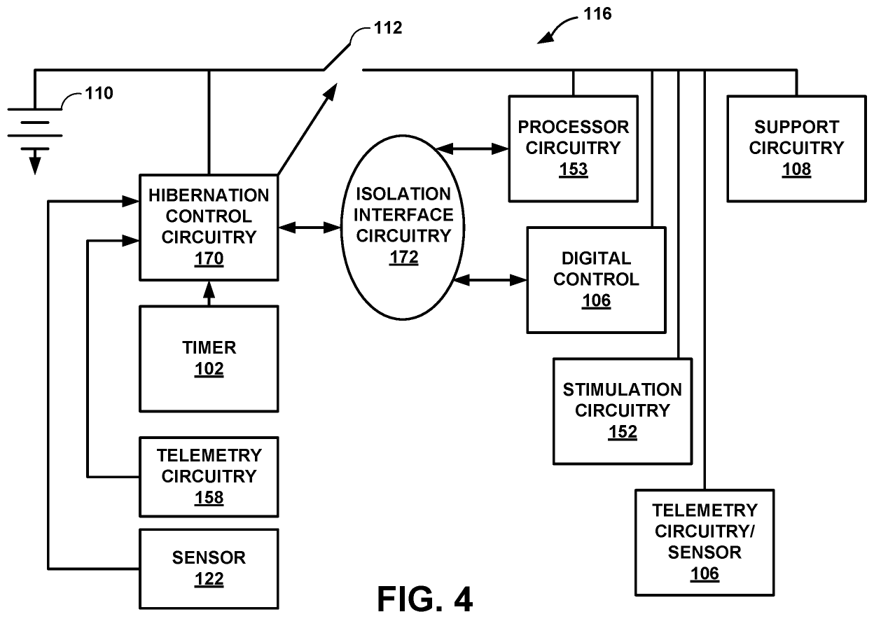

Hibernation control circuitry 70 may be configured to remove power normally provided by power source 60 from various components of IMD 32. In some examples, processor circuitry may be configured to determine whether IMD 32 should enter hibernation mode or not, and in response cause hibernation control circuitry 70 and/or isolation interface circuitry 72 to perform respective operations. For example, hibernation control circuitry 70 may be configured to remove power from processor circuitry 53, stimulation circuitry 52, and/or impedance circuitry 54. Hibernation control circuitry 70 may also be configured to restore power to such components upon exiting the hibernation mode. Isolation interface circuitry 72 may be configured to, while IMD 32 is in hibernation mode, prevent power from hibernation control circuitry 70 or other powered circuitry of IMD 32 leaking to, otherwise, unpowered components. A more detailed discussion of hibernation control circuitry 70 and isolation interface circuitry 72 follows with respect to FIGS. 4-7.

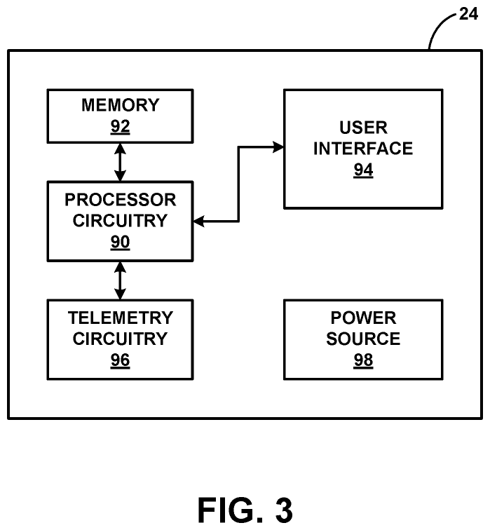

FIG. 3 is a block diagram illustrating an example configuration of an external programmer 24. While external programmer 24 may generally be described as a hand-held computing device, external programmer 24 may be a notebook computer, a smart phone, or a workstation, for example. As illustrated in FIG. 3, external programmer 24 may include a processor circuitry 90, memory 92, user interface 94, telemetry circuitry 96, and power source 98. Memory 92 may store program instructions that, when executed by processor circuitry 90, cause processor circuitry 90 and external programmer 24 to provide the functionality ascribed to external programmer 24 throughout this disclosure.

In general, external programmer 24 comprises any suitable arrangement of hardware, alone or in combination with software and/or firmware, to perform the techniques attributed to external programmer 24, and processor circuitry 90, user interface 94, and telemetry circuitry 96 of external programmer 24. In various examples, external programmer 24 may include one or more processors, such as one or more microprocessors, DSPs, ASICs, FPGAs, or any other equivalent integrated or discrete logic circuitry, as well as any combinations of such components. External programmer 24 also, in various examples, may include a memory 92, such as RAM, ROM, PROM, EPROM, EEPROM, flash memory, a hard disk, a CD-ROM, comprising executable instructions for causing the one or more processors to perform the actions attributed to them. Moreover, although processor circuitry 90 and telemetry circuitry 96 are described as separate circuitry, in some examples, processor circuitry 90 and telemetry circuitry 96 are functionally integrated. In some examples, processor circuitry 90 and telemetry circuitry 96 and telemetry circuitry 58 correspond to individual hardware units, such as microprocessors, ASICs, DSPs, FPGAs, or other hardware units. In other examples, any of processor circuitry 90 and telemetry circuitry 96 and telemetry circuitry 58 may correspond to multiple individual hardware units, such as microprocessors, ASICs, DSPs, FPGAs, or other hardware units.

Memory 92 may store program instructions that, when executed by processor circuitry 90, cause processor circuitry 90 and external programmer 24 to provide the functionality ascribed to external programmer 24 throughout this disclosure. In some examples, memory 92 may further include program information, e.g., stimulation programs defining the neurostimulation, similar to those stored in memory 56 of IMD 32. The stimulation programs stored in memory 92 may be downloaded into memory 56 of IMD 32.

In certain examples, the system includes a user interface 94 that allows patient 14 to provide input. IMD 32 may respond to patient-supplied data from the user interface by altering therapy. For example, patient 14 may use external programmer 24 (e.g., a handheld device) to record (by pushing a button) a physiological event of interest. Processor circuitry 53 of IMD 32 may respond by turning the therapy on or off and exiting or entering hibernation mode, or by adjusting the therapy (e.g., the stimulation strength) or by changing the therapy program. With reference to the urological applications discussed herein, patient 14 could push a button on external programmer 24 (e.g., their smartphone) when the bladder is voided. For example, this may send a signal to IMD 32 to turn off and enter hibernation mode for a period of time that is either pre-programmed based on the voiding characteristics of patient 14.

User interface 94 may include a button or keypad, lights, a speaker for voice commands, a display, such as a liquid crystal (LCD), light-emitting diode (LED), or cathode ray tube (CRT). In some examples the display may be a touch screen. As discussed in this disclosure, processor circuitry 90 may present and receive information relating to electrical stimulation and resulting therapeutic effects via user interface 94. For example, processor circuitry 90 may receive patient input via user interface 94. The input may be, for example, in the form of pressing a button on a keypad or selecting an icon from a touch screen.

Processor circuitry 90 may also present information to patient 14 in the form of alerts related to delivery of the electrical stimulation to patient 14 or a caregiver, as described in more detail below, via user interface 94. Although not shown, external programmer 24 may additionally or alternatively include a data or network interface to another computing device, to facilitate communication with the other device, and presentation of information relating to the electrical stimulation and therapeutic effects after termination of the electrical stimulation via the other device.

Telemetry circuitry 96 supports wireless communication between IMD 32 and external programmer 24 under the control of processor circuitry 90. Telemetry circuitry 96 may also be configured to communicate with another computing device via wireless communication techniques, or direct communication through a wired connection. In some examples, telemetry circuitry 96 may be substantially similar to telemetry circuitry 58 of IMD 32 described above, providing wireless communication via a radio frequency or proximal inductive medium. In some examples, telemetry circuitry 96 may include an antenna, which may take on a variety of forms, such as an internal or external antenna.

Examples of local wireless communication techniques that may be employed to facilitate communication between external programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Power source 98 delivers operating power to the components of programmer 24. Power source 98 may include a battery and a power generation circuit to produce the operating power. In some examples, the battery may be rechargeable to allow extended operation.

FIG. 4 is a block diagram illustrating an example neurostimulation device having a hibernation mode according to the techniques of this disclosure. IMD 116 includes battery 110 (which may be an example of power source 60 of FIG. 2), hibernation control circuitry 170 (which may be an example of hibernation control circuitry 70 of FIG. 2), timer 102, telemetry circuitry 158 (which may be an example of telemetry circuitry 58 of FIG. 2), and sensor 122 (which may be an example of sensor 22 of FIG. 2) on one side of isolation interface circuitry 172. IMD 116 includes processor circuitry 153 (which may be an example of processor circuitry 53 of FIG. 2), digital control 106, stimulation circuitry 152 (which may be an example of stimulation circuitry 52 of FIG. 2), and support circuitry 108 on another side of isolation interface circuitry 172. In some examples, IMD 116 also includes telemetry circuitry/sensor 106. Telemetry circuitry/sensor 106 may be telemetry circuitry and/or one or more sensors. In examples where telemetry circuitry/sensor 106 includes telemetry circuitry, such telemetry circuitry may be configured to use more power than telemetry circuitry 158 when in operation. For example, the telemetry circuitry of telemetry circuitry/sensor 106 may handle both uplink and downlink communications, while telemetry circuitry 158 may detect downlink communications. In examples where telemetry circuitry/sensor 106 includes one or more sensors, the one or more sensors may be sensors that are not required to be powered while IMD 116 is in hibernation mode. IMD 116 also includes switch 112.

When switch 112 is closed, battery 110 powers all of the circuitry of IMD 116 requiring power and IMD 116 may be in an operational mode. When switch 112 is open, battery 110 does not deliver power to processor circuitry 153, digital control 106, stimulation circuitry 152, telemetry circuitry/sensor 106, or support circuitry 108.

Hibernation control circuitry 170 is configured to control switch 112 to open or to close. When hibernation control circuitry 170 opens switch 112, power is removed from processor circuitry 153, digital control 106, stimulation circuitry 152, telemetry circuitry/sensor 106, and support circuitry 108. When hibernation control circuitry 170 closes switch 112, power is restored to processor circuitry 153, digital control 106, stimulation circuitry 152, telemetry circuitry/sensor 106, and support circuitry 108.

Hibernation control circuitry 170 or processor circuitry 153 may be configured to configure isolation interface circuitry 172 to prevent power leakage from powered components, such as hibernation control circuitry 170 and timer 102 to processor circuitry 153, digital control 106, stimulation circuitry 152, telemetry circuitry/sensor 106, and support circuitry 108 when IMD 116 is in hibernation mode. For example, hibernation control circuitry 170 may be configured to cause IMD 116 to enter a hibernation mode in response to a hibernation trigger and exit the hibernation mode in response to a wake-up trigger. For example, the hibernation trigger may be the end of a period of delivery of stimulation or the beginning of a period of withholding of stimulation (or a signal associated therewith, such as a signal from timer 102 indicative of the end of stimulation or the beginning of the withholding of stimulation), a signal from telemetry circuitry 158, or a signal from sensor 122, and the wake-up trigger may be a signal from timer 102, telemetry circuitry 158, or sensor 122.

Timer 102 may be configured to count down a programmable time period (e.g., on-cycle or off-cycle) and at the end of the programmable time period send a signal to hibernation control circuitry 170 indicative of the programmable time period having expired. This signal may be a wake-up trigger or, in some examples, a hibernation trigger. The programmable time period may have a lower limit and an upper limit within which a user can program the time period. For example, the lower limit may be less than one second and the upper limit may be more than one year. In some examples, the upper limit may be several years, such as ten years. In some examples, there may be no upper limit. The lower limit may be useful for preventing the quick cycling of IMD 116 between hibernation mode and operational mode as there may be power costs associated with the transition between the two modes.

In some examples, IMD 116 may be programmed to enter hibernation mode for a period of time during which delivery of stimulation is undesirable, such as during a pregnancy of patient 14. A user may program the programmable time period using external programmer 24. For example, a clinician may select a time period between the lower limit and the upper limit (if there is one), inclusive, via user interface 94 (FIG. 3). Hibernation control circuitry 170 may be configured to respond to the signal from timer 102 indicative of the end of the programmable time period by closing switch 112 to restore power to processor circuitry 153, digital control 106, stimulation circuitry 152, telemetry circuitry/sensor 106, and support circuitry 108.

Telemetry circuitry 158 may function similar to telemetry circuitry 58 of FIG. 2. In some examples, telemetry circuitry 158 may be continuously powered by battery 110. In some examples, telemetry circuitry 158 may be disconnected from battery 110 for periods of time while IMD 116 is in hibernation mode via a switch and timer (not shown). For example, telemetry circuitry 158 may be configured to wake up at a predetermined time interval, such as one time a minute or one time every ten minutes to advertise for a connection or to check for advertisements for a connection. When IMD 116 is in hibernation mode (e.g., switch 112 is open), if telemetry circuitry determines external programmer 24 (FIGS. 1 and 3) is attempting to establish communications with IMD 116, telemetry circuitry 158 may send a signal to hibernation control circuitry 170 indicative of external programmer 24 attempting to establish communications with IMD 116. In response to receiving the signal indicative of external programmer 24 attempting to establish communications with IMD 116, hibernation control circuitry 170 may close switch 112 to restore power to processor circuitry 153, digital control 106, stimulation circuitry 152, telemetry circuitry/sensor 106, and support circuitry 108, and IMD 116 may exit hibernation mode.

In some examples, if an active telemetry session is ongoing when IMD 116 would otherwise enter hibernation mode (e.g., a period of withholding of stimulation begins), processor circuitry 153 may prevent IMD 116 from entering hibernation mode until the active telemetry session ends. For example, processor circuitry 153 may monitor telemetry circuitry 158 to determine whether or not an active telemetry session is occurring. In some examples, telemetry circuitry 158 is configured to inductively couple with external programmer 24. In some examples, telemetry circuitry 158 is configured to couple with external programmer 24 via radio frequency.

Sensor 122 may function similarly to sensor 22 or impedance circuitry 54 of FIG. 2. In some examples, sensor 122 may be continuously powered by battery 110. In some examples, sensor 122 may be disconnected from battery 110 for periods of time while IMD 116 is in hibernation mode via a switch and timer (not shown). For example, sensor 122 may be configured to wake up at a predetermined time interval, such as one time a minute or one time every ten minutes to sense one or more parameters of interest. In some examples, sensor 122 may turn off for periods of time and wake up to sense a one or more physiological parameters of patient 14, an activity level of patient 14, a patient posture, a magnetic field, or other parameter of interest.

In some examples, the one or more parameters of interest may include one or more physiological parameters of patient 14. For example, sensor 122 may be configured to sense one or more physiological parameters of patient 14 (FIG. 1). In some examples, the one or more physiological parameters of patient 14 are indicative of the bladder of patient 14 being in a certain stage of a fill cycle, e.g., 75% full, or indicative of a number or intensity of bladder contractions. In some examples, sensor 112 is configured to send a signal indicative of the one or more physiological parameters of patient 14 to hibernation control circuitry 170 upon sensing the one or more physiological parameters of patient 14. In some examples, sensor 112 is configured to send a signal indicative of the one or more physiological parameters of patient 14 reaching a predetermined threshold to hibernation control circuitry 170 upon determining the one or more physiological parameters of patient 14 has reached a predetermined threshold. In some examples, hibernation control circuitry 170 is configured to determine whether the one or more physiological parameters of patient 14 has reached the predetermined threshold based on the signal from sensor 122.

In some examples, sensor 122 may include an accelerometer or a magnetic sensor. In some examples, sensor 122 may be configured to send a signal indicative of an activity level of patient 14 or a posture of patient 14 to hibernation control circuitry 170. In some examples, sensor 122 may be configured to send a signal to hibernation control circuitry 170 upon sensing a change in a magnetic field. Such signals may be wake-up or hibernation triggers.

Hibernation control circuitry 170 may be configured to close switch 112 based on a received signal from sensor 122. For example, in response to receiving the signal from sensor 122, hibernation control circuitry 170 may close switch 112. In another example, in response to determining that one or more physiological parameters of patient 14 indicates that stimulation should be resumed, hibernation control circuitry 170 may close switch 112. For example, in response to determining that one or more physiological parameters of patient 14 has reached or exceeded a predetermined threshold, hibernation control circuitry 170 may close switch 112. In another example, in response to determining that a level of patient activity or a patient posture indicates that stimulation should resume, hibernation control circuitry may close switch 112.

In some examples, hibernation control circuitry 170 may be configured to close switch 112 in response to signals from any of timer 102, telemetry circuitry 158, or sensor 122. In other words, if any of timer 102, telemetry circuitry 158, or sensor 122 (or a signal from sensor 122) indicates that IMD 116 should exit the hibernation mode, hibernation control circuitry 170 may close switch 112. When hibernation control circuitry 170 closes switch 112, power is restored to processor circuitry 153, digital control 106, stimulation circuitry 152, telemetry circuitry/sensor 106, and support circuitry 108.

Processor circuitry 153 may function similarly to processor circuitry 53 of FIG. 2. Stimulation circuitry 152 may function similarly to stimulation circuitry 52 of FIG. 2. Digital control 106 may be configured to control the sequencing and timing of the stimulation pulses, along with diagnostic data collection, such as battery voltage measurements and electrode impedance measurements. Support circuitry 108 may be configured to support other operations of IMD 116.

Isolation interface circuitry 172 may provide isolation from power for processor circuitry 153, digital control, 106, stimulation circuitry 152, and support circuitry 108. For example, isolation interface circuitry 172 may include a series of digital logic cells that presents a high impedance to the unpowered circuitry while presenting appropriate logic level voltage to the powered circuitry of hibernation control circuitry 170. For example, isolation interface circuitry 172 may hold digital signal from processor circuitry 153 to hibernation control circuitry 170 at a constant voltage level after the power is removed from processor circuitry 153.

In some examples, a medical device, IMD 116, includes battery 110 configured to provide power to the medical device. The medical device may include stimulation circuitry 152 configured to generate an electrical stimulation signal. The medical device may include hibernation control circuitry 170 configured to cause the medical device to enter a hibernation mode in response to a hibernation trigger and exit the hibernation mode in response to a wake-up trigger. The medical device may include switch 112 configured to open in response to hibernation control circuitry 170 causing the medical device to enter a hibernation mode and close in response to hibernation control circuitry 170 causing the medical device to exit the hibernation mode. The medical device may include isolation interface circuitry 172 configured to prevent power leakage from hibernation control circuitry 170 to stimulation circuitry 152 when the medical device is in hibernation mode. In such examples, stimulation circuitry 152 is not powered by battery 110 when the medical device is in the hibernation mode.

In some examples, power consumption from battery 110 during hibernation mode is in 5 microamps or less. In some examples, power consumption from battery 110 during hibernation mode is less than 1 microamp. In some examples, the hibernation trigger is associated with stimulation circuitry 152 ceasing to generate the electrical stimulation signal. In some examples, the medical device includes telemetry circuitry 158, wherein the hibernation trigger includes a command to enter the hibernation mode received by telemetry circuitry 158 from external device 24 (FIGS. 1 and 3). In some examples, the wake-up trigger includes a timer trigger and wherein the medical device further includes timer 102 communicatively coupled to hibernation control circuitry 170 and configured to generate the timer trigger at the expiration of a predetermined time period, wherein isolation interface circuitry 172 is further configured to prevent power leakage from timer 102 to stimulation circuitry 152. In some examples, the predetermined time period is a programmable time period with a lower limit of under one second and an upper limit of more than one year.

In some examples, the wake-up trigger includes a signal received by telemetry circuitry 158 from external device 24. In some examples, the signal is a radio frequency signal or an inductive coupling signal. In some examples, the medical device includes sensor 122 communicatively coupled to hibernation control circuitry 170, and wherein the wake-up trigger includes a sensor signal from sensor 122. In some examples, sensor 122 is further configured to sense a one or more physiological parameters of patient 14, and wherein sensor 122 generates the sensor signal in response to the sensing of the one or more physiological parameters of patient 14. In some examples, sensor 122 comprises a magnetic sensor or an accelerometer. In some examples, hibernation control circuitry 170 is further configured to power up and power down telemetry circuitry 158 or sensor 122 when the medical device is in hibernation mode.

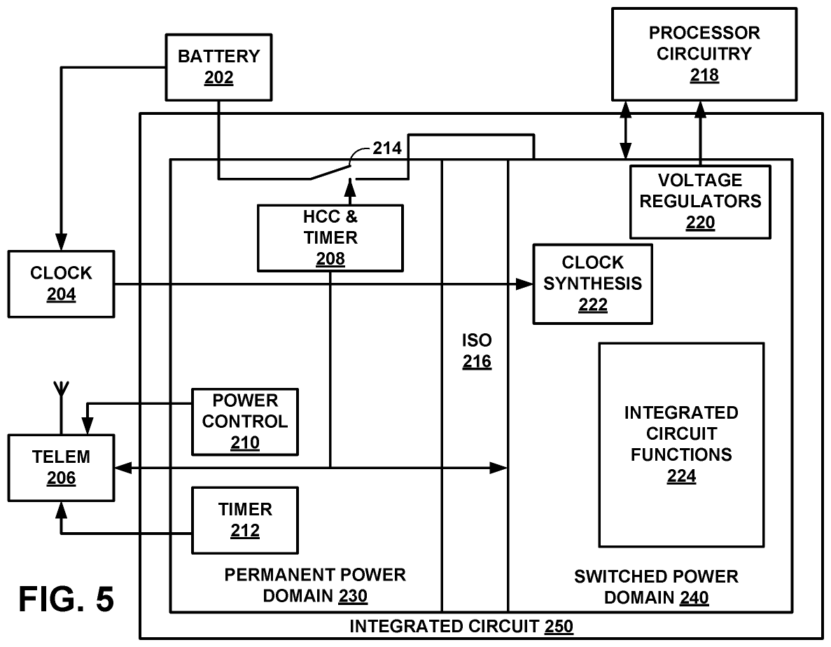

FIG. 5 is a block diagram illustrating example circuitry of a neurostimulation device according to the techniques of this disclosure. The example of FIG. 5 includes integrated circuit 250, battery 202 (which may be an example of power source 60 of FIG. 2 or battery 110 of FIG. 4), clock 204, telemetry circuitry 206 (shown as TELEM, which may be an example of telemetry circuitry 58 of FIG. 2 and telemetry circuitry 158 of FIG. 4), and processor circuitry 218 (which may be an example of processor circuitry 53 of FIG. 2 or processor circuitry 153 of FIG. 4). Clock 204 provides timing for the neurostimulation device. Integrated circuit 250 may include permanent power domain 230 which may be coupled to battery 202 as long as battery 202 is in the IMD, e.g., IMD 32 (FIGS. 1 and 3) or IMD 116 (FIG. 4). Integrated circuit 250 may also include switched power domain 240. Switched power domain 240 may be coupled to battery 202 while the IMD is in a normal operational mode and not be coupled to battery 202 when the IMD is in hibernation mode. Separating permanent power domain 240 and switched power domain 240 may be isolation interface circuitry 216 (shown as ISO 216). Isolation interface circuitry 216 may function similar to isolation interface circuitry 72 (FIG. 2) or isolation interface circuitry 172 (FIG. 4).

Permanent power domain 230 may include hibernation control circuitry and timer 208 (shown as HCC & TIMER 208) which may operate similarly to hibernation control circuitry 70 (FIG. 2), hibernation control circuitry 170 (FIG. 4), and timer 102 (FIG. 4). For example, hibernation control circuitry and timer 208 may be configured to open and close switch 214 (which may be an example of switch 112 of FIG. 4) when entering and exiting hibernation mode, respectively. When hibernation control circuitry and timer 208 opens switch 214, power is removed from processor circuitry 218, voltage regulators 220, clock synthesis 222, and integrated circuit functions 224. When hibernation control circuitry and timer 208 closes switch 214, power is restored to processor circuitry 218, voltage regulators 220, clock synthesis 222, and integrated circuit functions 224.

Permanent power domain 230 may also include power control circuitry 210 and timer 212. Power control circuitry 210 and timer 212 may be configured to power down telemetry circuitry 206 for intervals of time and power up telemetry circuitry 206 periodically to advertise for communications or to determine if external device 24 (FIGS. 1 and 3) is advertising for communications.

Clock 204 may provide timing for hibernation circuitry 200. In some examples, clock 204 may provide timing for the IMD. In some examples, clock 204 may be a crystal oscillator or another type of oscillator.

Switched power domain 250 may include integrated circuit functions 224, clock synthesis circuitry 222, and voltage regulators 220. Clock synthesis circuitry 222 may be configured to derive clock signals from clock 204 at appropriate frequencies for operations occurring within switched power domain 240. Voltage regulators 220 may be configured to provide power to processor circuitry 218 from switched power domain 250 when the IMD is in operational mode. By providing power to processor circuitry 218 from switched power domain 240, hibernation circuitry 200 may power down processor circuitry 218 when in hibernation mode by opening switch 214. Other components of the IMD not shown in FIG. 5 may also be powered through switched power domain 240. In this manner, when the IMD is in hibernation mode, power consumption by the IMD may be reduced to less than one microwatt.

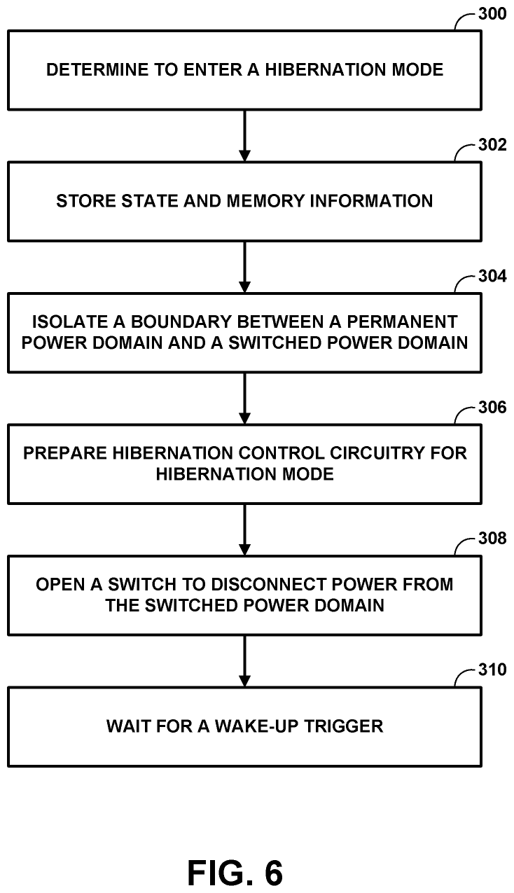

FIG. 6 is a flow diagram illustrating an example technique for entering a hibernation mode. While described with respect to IMD 116 of FIG. 4, the techniques of FIG. 6 may be performed by IMD 32 of FIGS. 1 and 3 or by hibernation circuitry 200 of FIG. 5 or a combination thereof.

Processor circuitry 153 may determine to enter a hibernation mode (300). For example, processor circuitry 153 may determine that a time period of withholding delivery of stimulation has been reached, such as from an active one of therapy programs 66 (FIG. 2). For example, processor circuitry 153 may determine to enter hibernation mode because the active stimulation program being used by IMD 116 exits a period of stimulation and enters a period of withholding of stimulation. In another example, processor circuitry 153 may receive a signal from timer 102 indicative of the beginning of a time period of withholding delivery of stimulation, or a sensor signal from sensor 122 indicative of stimulation ending or relating to a change in activity level, patient posture, or a one or more physiological parameters that processor circuitry 153 may determine indicates that stimulation should cease. In another example, telemetry circuitry 158 may receive a command to cease the delivery of stimulation from external programmer 24 (FIGS. 1 and 3). Telemetry circuitry 158 may provide the command to processor circuitry. In response to receiving the command, processor circuitry 153 may determine to enter hibernation mode. In this manner, processor circuitry 153 may determine to enter hibernation mode based on any one of the ceasing of the delivery of stimulation, a sensor signal, or a command received from external programmer 24. In some examples, processor circuitry 153 may be configured to be responsive to each of the ceasing of the delivery of stimulation (or a signal associated with the ceasing of the delivery of stimulation), a sensor signal, and a command received from external programmer to place IMD 116 into hibernation mode.

Processor circuitry 153 may store state and memory information in memory (302). For example, processor circuitry 153 may store state and memory information that may be needed when IMD 116 exits hibernation mode as processor circuitry 153 is going to be powered down while in hibernation mode.

Isolation interface circuitry 172 may isolate a boundary between a permanent power domain and a switched power domain (304). For example, hibernation control circuitry 170 or processor circuitry 153 may configure isolation interface circuitry 172 to isolate the boundary to prevent power leakage across the boundary. In this manner isolation interface circuitry 172 may form the boundary between the permanent power domain and the switched power domain.

Processor circuitry 153 may prepare hibernation control circuitry for hibernation mode (306). For example, processor circuitry 153 may signal hibernation control circuitry 170 to enter hibernation mode for a specific period of time. Hibernation control circuitry 170 may open switch 112 to disconnect power from the switched power domain (308). For example, hibernation control circuitry 170 may control switch 112 to open.

Hibernation control circuitry 170 may wait for a wake-up trigger (310). For example, hibernation control circuitry 170 may wait for a wake-up trigger signal from timer 102, telemetry circuitry 158, or sensor 122. In some examples, the wake-up trigger includes a timer signal from a timer. In some examples, timer 102 is configured to deliver the timer signal to hibernation control circuitry 172 at a programmable predetermined time period, wherein the programmable predetermined time period has a lower limit of under one second and an upper limit of more than one year. In some examples, the predetermined time period has no upper limit. In some examples, the wake-up trigger includes a signal received by telemetry circuitry 158. In some examples, the wake-up trigger includes a sensor signal.

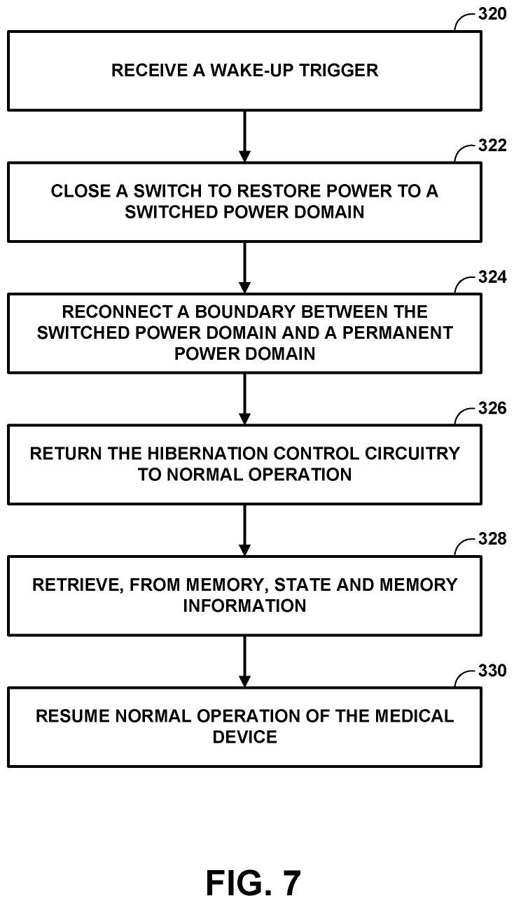

FIG. 7 is a flow diagram illustrating an example technique for exiting a hibernation mode. While described with respect to IMD 116 of FIG. 4, the techniques of FIG. 6 may be performed by IMD 32 of FIG. 2 or by hibernation circuitry 200 of FIG. 5 or a combination thereof.

Hibernation control circuitry 170 may receive a wake-up signal (320). For example, hibernation control circuitry 170 may receive a wake-up signal from timer 102, telemetry circuitry 158, or sensor 122.

Hibernation control circuitry 170 may close switch 112 to restore power to a switched power domain (e.g., switched power domain 240 of FIG. 5) (322). For example, hibernation control circuitry 170 may control switch 112 to close.

Processor circuitry 153 or hibernation control circuitry 170 may reconnect a boundary between the switched power domain and a permanent power domain (e.g., permanent power domain 230 of FIG. 5) (324). For example, processor circuitry 153 or hibernation control circuitry 170 may configure isolation interface circuitry 216 to reconnect operations between the switched power domain and the permanent power domain (e.g., reconnect clock 204 to clock synthesis 222 and reconnect telemetry circuitry 206 with integrated circuit functions 224, all of FIG. 5).

Processor circuitry 153 may return hibernation control circuitry 170 to normal operation (326). For example, processor circuitry 153 may reset hibernation control circuitry 170. Processor circuitry 153 may read hibernation information from memory 56 (FIG. 3), such as time spent in hibernation of a cause or origin of the wake-up signal (e.g., from timer 102, telemetry circuitry 158, or sensor 122).

Processor circuitry 153 may retrieve, from memory, state and memory information (328). For example, processor circuitry 153 may read state and memory information from memory needed for operation of IMD 116 in operational mode.

Processor circuitry 153 may resume normal operation of IMD 116 (330). For example, processor circuitry 153 may control stimulation circuitry 152 to begin delivering stimulation to patient 14.

In some examples, the wake-up signal comprises a timer signal from timer 102, a telemetry signal from telemetry circuitry 158, or a sensor signal from sensor 122. In some examples, timer 102 is configured to deliver the timer signal to hibernation control circuitry 170 at a programmable predetermined time period, wherein the programmable predetermined time period has a lower limit of under one second and an upper limit of more than one year.

It should be noted that system 10, and the techniques described herein, may not be limited to treatment or monitoring of a human patient. In alternative examples, system 10 may be implemented in non-human patients, e.g., primates, canines, equines, pigs, and felines. These other animals may undergo clinical or research therapies that my benefit from the subject matter of this disclosure.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processor circuitry, as well as any combinations of such components, remote servers, remote client devices, or other devices. The term “processor circuitry” or “processor circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processor circuitry, programmable processor circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

This disclosure includes the following non-limiting examples.

Example 1. A medical device comprising: a battery configured to provide power to the medical device; stimulation circuitry configured to generate an electrical stimulation signal; hibernation control circuitry configured to cause the medical device to enter a hibernation mode in response to a hibernation trigger and exit the hibernation mode in response to a wake-up trigger; a switch configured to open in response to the hibernation control circuitry causing the medical device to enter a hibernation mode and close in response to the hibernation control circuitry causing the medical device to exit the hibernation mode; and isolation interface circuitry configured to prevent power leakage from the hibernation control circuitry to the stimulation circuitry when the medical device is in hibernation mode, wherein the stimulation circuitry is not powered by the battery when the medical device is in the hibernation mode.

Example 2. The medical device of example 1, wherein power consumption from the battery during hibernation mode is less than 1 microamp.

Example 3. The medical device of example 1 or example 2, wherein the hibernation trigger is associated with the stimulation circuitry ceasing to generate the electrical stimulation signal.

Example 4. The medical device of example 1 or example 2, further comprising telemetry circuitry, wherein the hibernation trigger comprises a command to enter the hibernation mode received by the telemetry circuitry from the external device.

Example 5. The medical device of example 4, wherein the wake-up trigger comprises a timer trigger and wherein the medical device further comprises a timer communicatively coupled to the hibernation control circuitry and configured to generate the timer trigger at the expiration of a predetermined time period, wherein the isolation interface circuitry is further configured to prevent power leakage from the timer to the stimulation circuitry.

Example 6. The medical device of example 5, wherein the predetermined time period is a programmable time period with a lower limit of under one second and an upper limit of more than one year.

Example 7. The medical device of any of example 4, wherein the wake-up trigger comprises a signal received by the telemetry circuitry from the external device.

Example 8. The medical device of example 7, wherein the signal is a radio frequency signal or an inductive coupling signal.

Example 9. The medical device of any of examples 1-4, further comprising a sensor communicatively coupled to the hibernation control circuitry, and wherein the wake-up trigger further comprises a sensor signal from the sensor.

Example 10. The medical device of example 9, wherein the sensor is further configured to sense a one or more physiological parameters of a patient, and wherein the sensor generates the sensor signal in response to the sensing of the one or more physiological parameters of the patient.

Example 11. The medical device of example 9 or example 10, wherein the sensor comprises a magnetic sensor or an accelerometer.

Example 12. The medical device of any of example 1-11, wherein the hibernation control circuitry is further configured to power up and power down telemetry circuitry or a sensor when the medical device is in hibernation mode.

Example 13. A method of operating a medical device, the method comprising: determining, by processor circuitry, to enter a hibernation mode; storing, by the processor circuitry and in memory, state and memory information; isolating, by isolation interface circuitry, a boundary between a permanent power domain and a switched power domain; preparing, by the processor circuitry, hibernation control circuitry for hibernation mode; opening, by the hibernation control circuitry, a switch to disconnect power from the switched power domain; and waiting, by the hibernation control circuitry, for a wake-up trigger.

Example 14. The method of example 13, wherein the wake-up trigger comprises a timer signal from a timer.

Example 15. The method of example 14, wherein the timer is configured to deliver the timer signal to the hibernation control circuitry at a programmable predetermined time period, wherein the programmable predetermined time period has a lower limit of under one second and an upper limit of more than one year.

Example 16. The method of example 13, wherein the wake-up trigger comprises a signal received by telemetry circuitry.

Example 17. The method of example 13, wherein the wake-up trigger comprises a sensor signal.

Example 18. A method of operating a medical device, the method comprising: receiving, by hibernation control circuitry, a wake-up trigger; closing, by the hibernation control circuitry, a switch to restore power to a switched power domain; reconnecting, by isolation interface circuitry, a boundary between the switched power domain and a permanent power domain; returning, by processor circuitry, the hibernation control circuitry to normal operation; retrieving, by the processor circuitry from memory, state and memory information; and resuming, by the processor circuitry, normal operation of the medical device.

Example 19. The method of example 18, wherein the wake-up signal comprises a timer signal from a timer, a telemetry signal from telemetry circuitry, or a sensor signal from a sensor.

Example 20. The method of example 19, wherein the timer is configured to deliver the timer signal to the hibernation control circuitry at a programmable predetermined time period, wherein the programmable predetermined time period has a lower limit of under one second and an upper limit of more than one year.

Various examples have been described herein. Any combination of the described operations or functions is contemplated. These and other examples are within the scope of the following claims. Based upon the above discussion and illustrations, it is recognized that various modifications and changes may be made to the disclosed examples in a manner that does not require strictly adherence to the examples and applications illustrated and described herein. Such modifications do not depart from the true spirit and scope of various aspects of the disclosure, including aspects set forth in the claims.

What is claimed is:

1. A medical device comprising:
a battery configured to provide power to the medical device;
stimulation circuitry configured to generate an electrical stimulation signal;
hibernation control circuitry configured to cause the medical device to enter a hibernation mode in response to a hibernation trigger and exit the hibernation mode in response to a wake-up trigger;
a switch configured to open in response to the hibernation control circuitry causing the medical device to enter a hibernation mode and close in response to the hibernation control circuitry causing the medical device to exit the hibernation mode; and
isolation interface circuitry configured to prevent power leakage from the hibernation control circuitry to the stimulation circuitry when the medical device is in hibernation mode,
wherein the stimulation circuitry is not powered by the battery when the medical device is in the hibernation mode.

2. The medical device of claim 1, wherein power consumption from the battery during hibernation mode is less than 1 microamp.

3. The medical device of claim 1, wherein the hibernation trigger is associated with the stimulation circuitry ceasing to generate the electrical stimulation signal.

4. The medical device of claim 1, further comprising telemetry circuitry, wherein the hibernation trigger comprises a command to enter the hibernation mode received by the telemetry circuitry from the external device.

5. The medical device of claim 4, wherein the wake-up trigger comprises a timer trigger and wherein the medical device further comprises a timer communicatively coupled to the hibernation control circuitry and configured to generate the timer trigger at the expiration of a predetermined time period, wherein the isolation interface circuitry is further configured to prevent power leakage from the timer to the stimulation circuitry.

6. The medical device of claim 5, wherein the predetermined time period is a programmable time period with a lower limit of under one second and an upper limit of more than one year.

7. The medical device of claim 4, wherein the wake-up trigger comprises a signal received by the telemetry circuitry from the external device.

8. The medical device of claim 7, wherein the signal is a radio frequency signal or an inductive coupling signal.

9. The medical device of claim 1, further comprising a sensor communicatively coupled to the hibernation control circuitry, and wherein the wake-up trigger further comprises a sensor signal from the sensor.

10. The medical device of claim 9, wherein the sensor is further configured to sense a one or more physiological parameters of a patient, and wherein the sensor generates the sensor signal in response to the sensing of the one or more physiological parameters of the patient.

11. The medical device of claim 9, wherein the sensor comprises a magnetic sensor or an accelerometer.

12. The medical device of claim 1, wherein the hibernation control circuitry is further configured to power up and power down telemetry circuitry or a sensor when the medical device is in hibernation mode.

13. A method of operating a medical device, the method comprising:
determining, by processor circuitry, to enter a hibernation mode;
storing, by the processor circuitry and in memory, state and memory information;
isolating, by isolation interface circuitry, a boundary between a permanent power domain and a switched power domain;
preparing, by the processor circuitry, hibernation control circuitry for hibernation mode;
opening, by the hibernation control circuitry, a switch to disconnect power from the switched power domain; and
waiting, by the hibernation control circuitry, for a wake-up trigger.

14. The method of claim 13, wherein the wake-up trigger comprises a timer signal from a timer.

15. The method of claim 14, wherein the timer is configured to deliver the timer signal to the hibernation control circuitry at a programmable predetermined time period, wherein the programmable predetermined time period has a lower limit of under one second and an upper limit of more than one year.

16. The method of claim 13, wherein the wake-up trigger comprises a signal received by telemetry circuitry.

17. The method of claim 13, wherein the wake-up trigger comprises a sensor signal.

18. A method of operating a medical device, the method comprising:
receiving, by hibernation control circuitry, a wake-up trigger;
closing, by the hibernation control circuitry, a switch to restore power to a switched power domain;
reconnecting, by isolation interface circuitry, a boundary between the switched power domain and a permanent power domain;
returning, by processor circuitry, the hibernation control circuitry to normal operation;
retrieving, by the processor circuitry from memory, state and memory information; and
resuming, by the processor circuitry, normal operation of the medical device.

19. The method of claim 18, wherein the wake-up signal comprises a timer signal from a timer, a telemetry signal from telemetry circuitry, or a sensor signal from a sensor.

20. The method of claim 19, wherein the timer is configured to deliver the timer signal to the hibernation control circuitry at a programmable predetermined time period, wherein the programmable predetermined time period has a lower limit of under one second and an upper limit of more than one year.
